(19) United States
(12) Patent Application Publication
(10) Pub. No.: US 2023/0145426 A1
(43) Pub. Date: May 11, 2023

(54) PARTICLES COMPRISING INULIN FOR USE AS MEDICAMENT OR SUPPLEMENT

(71) Applicant: WHOLEFIBER HOLDING B.V., Amersfoort (NL)

(72) Inventors: Frederik KAPER, Pennington, NJ (US); Willem DE VOS, Amsterdam (NL)

(21) Appl. No.: 17/917,595

(22) PCT Filed: Apr. 2, 2021
(86) PCT No.: PCT/EP2021/058788

§ 371 Date: Oct. 7, 2022

(30) Foreign Application Priority Data
Apr. 7, 2020 (EP) 20168391.9

(51) Int. Cl. C08B 37/00; A61K 31/733

(52) U.S. Cl. CPC C08B 37/0054; C08B 37/0003; A61K 31/733

(57) ABSTRACT

A vegetable product obtained from vegetable matter selected from at least one of root, tubers and leaves of a plant having inulin as a reserve carbohydrate, wherein the product includes particles having particle sizes in the range of 0.5 to 10 mm and including inulin and cell walls having pectin, hemi-cellulose and cellulose, wherein the inulin is contained within the cell walls, and the product is obtained by a process including the steps of:
a) wounding the vegetable matter,
b) treating the vegetable matter with an aqueous solution having an antioxidant and/or a textural support agent and heating the vegetable matter to a temperature of 40 to 90° C. and
c) mechanically reducing the size of the vegetable matter and drying the vegetable matter to obtain the product.

### FIELD OF THE INVENTION

The present invention relates to a product comprising inulin and a process for preparing such product. The invention further relates such product for use in a medicament and a dietary supplement.

### BACKGROUND OF THE INVENTION

The highest levels of inulin are found in plant roots and tubers and reportedly the highest inulin content is to be found in the roots of chicory (Chicorium intybus L.) where up to 75% of the dry weight consists of fructose polymers. Hence, chicory roots are used as raw material for the industrial production of the food ingredient inulin. This inulin is extracted and purified from the plant cells after extensive processing treatments resulting in a pure fructan polymers that are produced as a powder and used as a food ingredient (Van Loo J et al, Crit Rev Food Sci Nutr. 1995 November; 35(6):525-52).

A product comprising inulin is further known from WO2014/172486, which discloses a method of manufacturing a high fiber vegetable product by inhibiting production of bitter components produced in vegetable matter of a plant having inulin as a reserve carbohydrate. The method comprises bathing the vegetable matter with water including an antioxidant; heating the vegetable matter to a temperature of 40° C. to 90° C.; and wounding (mechanically processing) the vegetable matter. The method may further comprise homogenizing the mechanically processed matter to obtain a suspension. This suspension may be dried to obtain a high fiber vegetable product and further processing it into e.g. fiber rich powder. The size of such powder is not mentioned.

While the high fiber vegetable product of WO2014/172486 has various advantages, there is a need for a product which has even more beneficial health effects.

### SUMMARY OF THE INVENTION

It is an objective of the present invention to provide a vegetable product in which the above-mentioned and/or other needs are met.

Accordingly, the present invention provides a vegetable product obtained from vegetable matter selected from at least one of root, tubers and leaves of a plant having inulin as a reserve carbohydrate, wherein the product comprises particles having particle sizes in the range of 0.5 to 10 mm and comprising inulin and cell walls comprising pectin, hemicellulose and cellulose, wherein the inulin is contained within the cell walls, and the product is obtained by a process comprising the steps of:
a) wounding the vegetable matter,
b) treating the vegetable matter with an aqueous solution comprising an antioxidant and/or a textural support agent and heating the vegetable matter to a temperature of 40 to 90° C. and
c) mechanically reducing the size of the vegetable matter and drying the vegetable matter to obtain the product.

According to the invention, the product is obtained by a process by which the majority of the cell walls of the vegetable matter remain intact such that inulin remains inside the cell walls, instead of the known process for preparing purified inulin powder by which inulin is extracted from the cell walls.

Step a) is a pre-treatment step of step b) for removing bitter components from the vegetable matter and is performed in such a way that major part of the cells of the vegetable matter remains intact.

Step c) is performed such that the obtained product comprises particles having sizes in the range of 0.5 to 10 mm. The obtained product may further comprise particles having sizes outside the range of 0.5 to 10 mm. However, preferably majority of product, i.e. at least 50 weight (wt) % of the product according to the invention, are the particles having sizes of 0.5 to 10 mm. More preferably, at least 60 wt. %, at least 70 wt. %, at least 80 wt. %, at least 90 wt. %, at least 95 wt. %, at least 98 wt. %, at least 99 wt. % or 100 wt. % of the product according to the invention are the particles having sizes of 0.5 to 10 mm.

This may be achieved by appropriately selecting the degree of the mechanical size reduction and optionally treating the obtained product to remove particles with sizes outside of the desired range. Various commercial equipment for creating fractions of desired particle sizes (e.g. sieving, air separation) are known to the skilled person.

The term ‘particle size’ is herein understood as the largest dimension of the particle, which can be determined by visual inspection of the particle. Visual inspection includes here also microscopic and electron microscopic analysis.

### BRIEF DESCRIPTION OF THE DRAWINGS

FIG. 1 shows photograph images of ITF and WF1-WF3, prior to the incubation; and

FIG. 2 shows the results in photograph images.

### DETAILED DESCRIPTION OF THE INVENTION

The inventors have surprisingly found that the selection of the relatively large size of the particles leads to various health benefits. An unexpected swelling in a simulated gastric compartment was observed for the particles according to the invention having a certain, relatively large size. The swelling of the product leads to a decrease in intestinal passage pace, leading to prolonged feelings of satiety. Such swelling was not observed with the extracted, purified powder consisting essentially of inulin. It was observed that larger particles led to a higher degree of swelling.

The product according to the invention generates short chain fatty acids (SCFAs) at a similar level and kinetics as smaller particles or the purified powder consisting essentially of inulin, as observed by a simulated colon model.

The product according to the invention generates significantly higher levels of butyrate, resulting in a significantly higher ratio of butyrate over propionate and butyrate over acetate, compared to the purified powder consisting essentially of inulin, as observed in a simulated colon model. It was also observed that larger particles result in a higher ratio of butyrate over propionate and butyrate over acetate than smaller particles. This indicates superior health benefits of the product of the invention since it has been well established that colonic butyrate has a panoply of health promoting functions.

The product according to the invention stays relatively intact in the colon and hence may reach the distal colon and at that site generate SCFA and notably butyrate, as observed by a simulated colon model. It was also observed that larger particles have a higher possibility of reaching the distal colon. It has been shown that distal colonic production of SCFA is highly beneficial.

The product according to the invention generates significantly less gas than the purified powder consisting essentially of inulin, as observed by a simulated colon model, which is highly relevant for the applicability of these products. It was also observed that larger particles produce less gas.

Product

The product according to the invention is made from a vegetable matter which is one or more of root, tubers and leaves of a plant having inulin as a reserve carbohydrate. The vegetable matter may derive from a plant belonging to the Asteraceae family. The plant may be but is not limited to chicory (Cichorium intybus var. sativum), globe artichoke (Cynara scolymus), Jerusalem artichoke (Helianthus tuberosus), endive (Cichorium endive), Belgian endive roots (Cichorium intybus var. foliosum), dandelion (Taraxacum officinale), dahlia (dahlia ssp.), burdock (Arctium lappa), salsify (Tragopogon porrifolius), and yacon (Smallanthus sonchifolius). The vegetable matter may be derived from any agricultural crop with a high concentration of inulin and/or other vegetable mater comprising inulin at an amount of at least 15 wt. % with respect to the vegetable mater in a non-dried state.

Preferably, the plant is chicory or Jerusalem artichoke.

The particles in the product according to the invention comprise cell walls comprising pectin, hemi-cellulose and cellulose and inulin contained within the cell walls. This can be determined e.g. by a microscope.

Preferably, the amount of inulin with respect to the product is at least 50 wt. %, for example 60 to 80 wt. %.

The product may further comprise at least one substance selected from the group consisting of lignin, protein, mono- and disaccharides and potassium.

In some embodiments, the total amount of inulin, pectin, hemicellulose and cellulose with respect to the product is at least 80 wt. %.

In some embodiments, the amount of inulin with respect to the product is 60 to 75 wt. %, e.g. 68 to 75 wt. %, the amount of pectin with respect to the product is 5 to 12 wt. %, e.g. 7 to 12 wt. % and the total amount of hemi-cellulose and cellulose with respect to the product is 4 to 8 wt. %.

In some embodiments, the product comprises protein in an amount of 4 to 7 wt. % with respect to the product.

In some embodiments, the product comprises mono- and disaccharides in an amount of 1 to 10 wt % of the product.

In some embodiments, the product comprises potassium in an amount of 0.5 to 2 wt % of the product.

Size of Particles

Preferably, at least 50 wt. % 60 wt. %, at least 70 wt. %, at least 80 wt. %, at least 90 wt. %, at least 95 wt. %, at least 98 wt. %, at least 99 wt. % or 100 wt % of the product according to the invention are the particles having particle sizes in the range of 0.5 to 10 mm and comprising inulin and cell walls comprising pectin, hemi-cellulose and cellulose, wherein the inulin is contained within the cell walls.

Preferably, said particles having particle sizes in the range of 0.5 to 10 mm comprise particles having sizes in the range of 1.0 to 10 mm. More preferably, the particles having particle sizes in the range of 0.5 to 10 mm comprise particles having sizes in the range of 1.5 to 8.0 mm. In some embodiments, the particles having particle sizes in the range of 0.5 to 10 mm may comprise particles having sizes in the range of 1.5 to 4.0 mm. In some embodiments, the particles having particle sizes in the range of 0.5 to 10 mm may comprise particles having sizes in the range of 4.0 to 8.0 mm.

Preferably, at least 50 wt. % 60 wt. %, at least 70 wt. %, at least 80 wt. %, at least 90 wt. %, at least 95 wt. %, at least 98 wt. %, at least 99 wt. % or 100 wt. % of the product according to the invention are the particles having particle sizes in the range of 1.0 to 10 mm and comprising inulin and cell walls comprising pectin, hemi-cellulose and cellulose, wherein the inulin is contained within the cell walls.

Preferably, at least 50 wt. % 60 wt. %, at least 70 wt. %, at least 80 wt. %, at least 90 wt. %, at least 95 wt. %, at least 98 wt. %, at least 99 wt. % or 100 wt. % of the product according to the invention are the particles having particle sizes in the range of 1.5 to 8.0 mm and comprising inulin and cell walls comprising pectin, hemi-cellulose and cellulose, wherein the inulin is contained within the cell walls.

Preferably, at least 50 wt. % 60 wt. %, at least 70 wt. %, at least 80 wt. %, at least 90 wt. %, at least 95 wt. %, at least 98 wt. %, at least 99 wt. % or 100 wt. % of the product according to the invention are the particles having particle sizes in the range of 1.5 to 4.0 mm and comprising inulin and cell walls comprising pectin, hemi-cellulose and cellulose, wherein the inulin is contained within the cell walls.

Preferably, at least 50 wt. % 60 wt. %, at least 70 wt. %, at least 80 wt. %, at least 90 wt. %, at least 95 wt. %, at least 98 wt. %, at least 99 wt. % or 100 wt. % of the product according to the invention are the particles having particle sizes in the range of 4.0 to 8.0 mm and comprising inulin and cell walls comprising pectin, hemi-cellulose and cellulose, wherein the inulin is contained within the cell walls.

The particles may have various shapes, e.g. close to spherical or polyhedron such as cuboid, cube etc, but preferably do not have a fibrous shape. In a fibrous shaped particle, the ratio between its largest dimension (normally referred as length) is very large with respect to its smallest dimension (normally referred as thickness). A fibrous shaped particle also has a low weight and a low volume with respect to its largest dimension.

Preferably, at least 50 wt. %, at least 60 wt. %, at least 70 wt. %, at least 80 wt. %, at least 90 wt. %, at least 95 wt. %, at least 98 wt. %, at least 99 wt. % or 100 wt. % of the product according to the invention are particles having a ratio between the largest dimension and the smallest dimension in the range of at most 4, at most 3 or at most 2.

Preferably, the particles have shapes which have a low surface area to volume ratio.

Preferably, at least 50 wt. %, at least 60 wt. %, at least 70 wt. %, at least 80 wt. %, at least 90 wt. %, at least 95 wt. %, at least 98 wt. %, at least 99 wt. % or 100 wt. % of the product according to the invention are particles having a surface area to volume (SA/V) ratio of at most 6 mm⁻¹, 5 mm⁻¹, 4 mm⁻¹, 3 mm⁻¹, 2 mm⁻¹ or 1 mm⁻¹.

The SA/V ratio of a particle may be calculated by approximating the particle as a cube or a sphere. Evidently, particles smaller than 200 micrometer (0.2 mm) have, when occurring as cubes with a side of 0.2 mm or as a sphere with a radius 0.1 mm), a (SA/V) ratio of over 30.

| side or | Surface/Volume | |
|---|---|---|
| radius | Ratio (mm⁻¹) | |
| in mm | cubes | spheres |
| 0.1 | 60 | 32.5 |
| 0.2 | 30 | 14.7 |
| 0.5 | 12 | 6 |
| 1 | 6 | 3 |
| 2 | 3 | 1.5 |
| 3 | 2 | 1 |
| 4 | 1.5 | 0.75 |
| 5 | 1.2 | 0.6 |
| 6 | 1 | 0.5 |
| 7 | 0.86 | 0.43 |
| 8 | 0.75 | 0.37 |

Process Steps

By steps a) and b) of the process according to the invention, bitter components produced in the vegetable matter are inactivated or removed or the production of the bitter components is inhibited. This is described in detail in WO2014/172486, incorporated herein by reference.

As used herein, the phrase “bitter components” refers to mainly sesquiterpenes. The sesquiterpenes may be sesquiterpene lactones produced by plants belonging to the Asteraceae family. Many plants of the Asteraceae family have ducts and/or trichomes, containing enzymes and/or precursors to form sesquiterpene lactones. These sesquiterpene lactones may be secreted by plants upon wounding of the plant organs or tissues. The bitter taste of the plants may be associated with the secreted sesquiterpene lactones. Sesquiterpene lactones may include but are not be limited to guaianolides, eudesmanolides, or germacranolides. The guaianolides may include at least one of lactucin, 8-deoxylactucin, and lactucopicrin.

Step a)

Wounding may involve cutting top or tail of the root, tuber or leaves of the vegetable matter or physically damaging it to facilitate the removal of the bitter components.

It will be appreciated that step a) does not damage the cell walls in the inside of the vegetable matter and the product obtained after step a) comprises cell walls containing inulin within the cell walls. It will also be appreciated that step a) is performed such that the size reduction caused by the wounding does not result in the vegetable matter too small to obtain the desired particle size.

Step b)

After step a), the vegetable matter is subjected to treating with an aqueous solution and heating.

The heating of the vegetable matter may be performed before, during and/or after the treating the vegetable matter with the aqueous solution. Preferably, the heating is performed during the treating the vegetable matter by bathing or dipping the vegetable matter in the aqueous solution heated to a temperature of 40° C. to 90° C., preferably 45 to 65° C. In other embodiments, the heating may be performed before and/or after the step of treating with the aqueous solution by bathing or dipping the vegetable matter in water heated to a temperature of 40° C. to 90° C.

The aqueous solution may have a temperature of more than 0 to 90° C., for example more than 0° C. and less than 40° C. or at least 40° C. and at most 90° C.

Treating with the aqueous solution may involve dipping the vegetable matter into the aqueous solution, bathing the vegetable matter with the aqueous solution, rinsing the vegetable matter with the aqueous solution or spraying the vegetable matter with the aqueous solution.

The aqueous solution comprises an antioxidant and/or a textural support agent.

The antioxidant may be an inhibitor of one or more enzymes involved in forming bitter sesquiterpene lactones. The inhibitor may be an acidulant, preferably, an organic acidulant. The organic acidulant may be but is not limited to ascorbic acid, citric acid, erythorbic acid, lactic acid, gluconic acid, malic acid or salts thereof. The salts thereof may be potassium salts or sodium salts. The salts may be but are not limited to at least one of potassium ascorbate, potassium citrate, potassium erythorbate, potassium lactate, potassium gluconate, sodium ascorbate, sodium citrate, sodium erythorbate, sodium lactate, or sodium gluconate, sodium malate.

The textural support agent may be but is not limited to CaCl₂, Ca-gluconate, Ca-lactate, Ca-lactate gluconate. The textural support agent may be used for treating and preserving texture of the vegetable matter.

The aqueous solution may further comprise an agent that inactivates sesquiterpene lactones. The agent may include sulfhydryl or sulfite groups. The agent may be but is not limited to L-cysteine, L-cysteine HCl, thiol containing peptides, papaya extract, one or more proteases or glycosidases, sodium bisulfite (NaHSO₃), or potassium bisulfite (KHSO₃).

The aqueous solution may further comprise a chelating or sequestering agent. The chelating or sequestering agent may bind or trap metal ions included in plant enzymes involved in the formation of bitter sesquiterpene lactones. The metal ions may be but are not limited to ions of Fe, Cu, Mg or Ca. The metal ions may be but are not limited to Fe²⁺, Fe³⁺, Cu⁺, Cu²⁺, Mg²⁺ or Ca²⁺. The chelating or sequestering agent may be but are not limited to ethylendiamine tetraacetic acid (EDTA), sodium pyrophosphate Na₄P₂O₇, potassium pyrophosphate (K₄P₂O₇), or sodium acid pyrophosphate (Na₂H₂P₂O₇).

The aqueous solution may further comprise a complexing agent. The complexing agent may entrap active sites responsible for bitterness of sesquiterpene lactones. The complexing agent may be but is not limited to cyclodextrin.

Preferably, the aqueous solution comprises at least one substance selected from the group consisting of: ascorbic acid, citric acid, erythorbic acid, lactic acid, gluconic acid, malic acid, potassium ascorbate, potassium citrate, potassium erythorbate, potassium lactate, potassium gluconate, sodium ascorbate, sodium citrate, sodium erythorbate, sodium lactate, sodium gluconate, sodium malate, cyclodextrin, sodium pyrophosphate, sodium acid pyrophosphate, potassium pyrophosphate, potassium acid pyrophosphate, L-cysteine, L-cysteine-HCL, thiol containing peptides, extract, proteases, glycosidases, sodium bisulfite, potassium bisulfite, EDTA, CaCl₂, Ca-lactate, Ca-gluconate, and Ca-lactate gluconate.

Step c)

After step b), the vegetable matter is mechanically treated such that the size of the vegetable matter is reduced. The vegetable matter is also dried. The drying step may be performed before, during and/or after the mechanical size reduction step. Preferably, the drying step is performed after the mechanical size reduction step. Step c) is performed such that the resulting particles have the desired particle sizes.

The mechanical size reduction may involve e.g. dicing, shredding and/or slicing.

Drying may include removing moisture e.g. by hot air having a temperature of e.g. 50 to 90° C.

Typically, the vegetable matter has a water content of e.g. 70 wt. % before drying, which is reduced to e.g. 8 wt. % after drying. The drying does not on its own (without the mechanical size reduction) substantially reduce the size of the vegetable matter. Thus, the density of the vegetable matter after drying is much lower than the density of the vegetable matter before drying and is very low. Accordingly, the vegetable product according to the invention may have a density of at most 0.9 g/cm³, at most 0.8 g/cm³, at most 0.7 g/cm³, at most 0.6 g/cm³ or at most 0.5 g/cm³. The vegetable product according to the invention may have a density of at least 0.05 g/cm³ or at least 0.1 g/cm³. The ratio of the density of the vegetable matter obtained after step b) to the vegetable product according to the invention may e.g. be 2 to 20, 3 to 15 or 5 to 10.

Optional Steps

The process may further comprise the step of washing the vegetable matter before step a).

The process may further comprise the step of storing the vegetable matter between steps a) and b) and/or between steps b) and c), e.g. at a temperature between −20° C. to 20° C.

The invention further relates to a process for making a vegetable product obtained from vegetable matter selected from at least one of root, tubers and leaves of a plant having inulin as a reserve carbohydrate, wherein the product comprises particles having particle sizes in the range of 1.0 to 10 mm and comprising inulin and cell walls comprising pectin, hemi-cellulose and cellulose, wherein the inulin is contained within the cell walls, and the process comprises the steps of:
a) wounding the vegetable matter,
b) treating the vegetable matter with an aqueous solution comprising an antioxidant and/or a textural support agent and heating the vegetable matter to a temperature of 40 to 90° C.,
c) mechanically reducing the size of the vegetable matter and drying the vegetable matter to obtain the product.

Product as Medicament or Dietary Supplement

The invention further relates to the use of the product according to the invention as a medicament. The invention further relates to the product according to the invention for use of as a medicament. The invention further relates to a medicament comprising the product according to the invention.

The invention further relates to the use of the product according to the invention as a dietary supplement. The invention further relates to the product according to the invention for use of as a food supplement. The invention further relates to a dietary supplement comprising the product according to the invention.

Preferably, the medicament is selected from the group consisting of:
Treatment of constipation and maintenance of normal defecation,
Treatment of metabolic and immune disorder selected from the group consisting of:
   metabolic syndrome,
   insulin-deficiency or insulin-resistance related disorders,
   pre-diabetes
   Diabetes Mellitus (such as, for example, Type 2 Diabetes),
   glucose intolerance,
   abnormal lipid metabolism,
   atherosclerosis,
   hypertension,
   cardiac pathology,
   stroke,
   non-alcoholic fatty liver disease,
   hyperglycemia,
   hepatic steatosis,
   dyslipidemia,
   dysfunction of the immune system associated with overweight and obesity,
   cardiovascular diseases,
   high cholesterol,
   elevated triglycerides,
   atherosclerosis,
   asthma,
   sleep apnoea,
   osteoarthritis,
   neuro-degeneration,
   gallbladder disease and
   cancer
Increase of vaccination efficiency,
Prevention of neurodegeneration,
Prevention of colon rectal cancer,
Increase of satiety,
Treatment and/or prevention of one or more of the following:
   Acute gastroenteritis
   Irritable bowel syndrome
   Aspecific chronic diarrhea
   Traveler's diarrhea
   Antibiotic associated diarrhea
   Acute gastroenteritis
   Inflammatory Bowel Disease (IBD) including Ulcerative colitis and Chrohn's disease
   Short bowel syndrome and intestinal failure
   Prevention of colorectal cancer
   Intestinal polyposis
   Pouchitis
   Allergic colitis and
Prevention of colorectal cancer
Treatment of distal colon cancer.

It has been well established that the human body lacks the enzymatic machinery to digest inulin, pectin or (hemi)cellulose i)cellulose and hence the caloric load of these fibers is very low. Moreover, these fibers will reach the colon in a predominantly unmodified form. Hence these fibers may be digested in the colon by the intestinal microbes, also termed microbiota, that colonize that part of the intestinal tract (Salonen et al, ISME J. 8: 2218-2230, 2014). In that anaerobic digestion process, these intestinal microbes generate among others short chain fatty acids (SCFAs), such as acetate, propionate and butyrate (Canfora et al, Sci Rep. 2019 Aug. 29; 9(1):12515). All SCFAs, particularly butyrate, are known to fuel enterocytes in the intestinal tract. However, the SCFAs are also taken up and have systemic effects on metabolic and immune health via the G-protein coupled receptors GPR41 and GPR43 (Schroeder & Backhed Nat Med. 2016 October; 22(10):1079-1089). Because of the health benefits of the SCFA's they produce, several of the fibers are known as prebiotics, which are defined as substrates that are selectively utilized by host microorganisms conferring a health benefit (Gibson et al Nat Rev Gastroenterol Hepatol. 2017 August; 14(8):491-502).

Animal studies have shown that increasing the SCFA availability and their signaling via the GPR43 receptor prevents diet-induced body weight gain, counteracts adiposity, and improves glucose homeostasis and insulin sensitivity (Brooks et al 2017). Moreover, acetate and to some extent propionate have been reported to reduce appetite (Frost et al Nature Comm. 2014; 5; Byrne et al Nutrients. 2019 Apr. 16; 11(4).). Importantly, butyrate also is recognized by the receptor GPR109a and functions as a tumor suppressor in the colon (Thangaraju et al Cancer Res. 2009 Apr. 1; 69(7):2826-32). Moreover, butyrate and to a lesser extent propionate, acts as a histone deacetylase (HDAC) inhibitor, providing anti-inflammatory and anti-tumor effects, whereas it is also involved in epigenetic programming (Liu et al Adv Nutr. 2018 Jan. 1; 9(1):21-29).

Recent human studies also showed that it is important at which location the SCFAs are produced in the colon. SCFAs were administered rectally and found to beneficially affect substrate and energy metabolism only when these were administered in the distal colon and not in the proximal colon (Canfora et al Sci Rep. 2017; 7(1):2360). Various beneficial effects were noted of the distal colonic infusions of SCFA mixtures, including increased fat oxidation, energy expenditure and satiety-stimulating hormones, as well as attenuated whole-body lipolysis. Hence, fibers that reach the distal colon and increase SCFAs production at this site, have a high potential for influencing host metabolism and metabolic health by improving adipose tissue function, preventing lipid overflow and skeletal muscle fat accumulation thereby improving insulin sensitivity (Canfora et al, Sci Rep. 2019 Aug. 29; 9(1):12515).

To predict the behavior of fibers in the human gastro-intestinal tract and assess potential health benefits use is made of model systems that simulate human gut microbiome, such as the Simulator of the Human Intestinal Microbial Ecosystem (SHIME) that has been used in a great number of studies (Marzorati et al BMC Microbiol. 2014 May 22; 14:133; van den Abbeele et al ISME J. 2013 May; 7(5):949-61). In this and other gastro-intestinal models, fibers are usually tested in anaerobic reactors where the behavior such as swelling can be monitored in a simulated upper part of the gastro-intestinal tract, after which short-term incubations are performed using fecal samples of human volunteers. In these latter colonic incubations, the formation of SCFAs and other products is determined as a function of time. One of these other products is gas that is formed as a side product of microbial fiber fermentation, and consists mainly of hydrogen but also carbon dioxide, methane or ammonia and the like. In the colon this gas production may lead to flatulence, belching or bloating, rumbling, cramps, distension, or other undesired discomforts.

According to the invention, a vegetable product is provided which can reach distal colon, which has a high capacity to lead to the production of SCFAs, notably butyrate, while and at the same time lead to a low level of intestinal gas production.

It is noted that the invention relates to the subject-matter defined in the independent claims alone or in combination with any possible combinations of features described herein, preferred in particular are those combinations of features that are present in the claims. It will therefore be appreciated that all combinations of features relating to the composition according to the invention; all combinations of features relating to the process according to the invention and all combinations of features relating to the composition according to the invention and features relating to the process according to the invention are described herein.

It is further noted that the term ‘comprising’ does not exclude the presence of other elements. However, it is also to be understood that a description on a product/composition comprising certain components also discloses a product/composition consisting of these components. The product/composition consisting of these components may be advantageous in that it offers a simpler, more economical process for the preparation of the product/composition. Similarly, it is also to be understood that a description on a process comprising certain steps also discloses a process consisting of these steps. The process consisting of these steps may be advantageous in that it offers a simpler, more economical process.

When values are mentioned for a lower limit and an upper limit for a parameter, ranges made by the combinations of the values of the lower limit and the values of the upper limit are also understood to be disclosed.

The invention is now elucidated by way of the following examples, without however being limited thereto.

Examples

Experimental Set 1

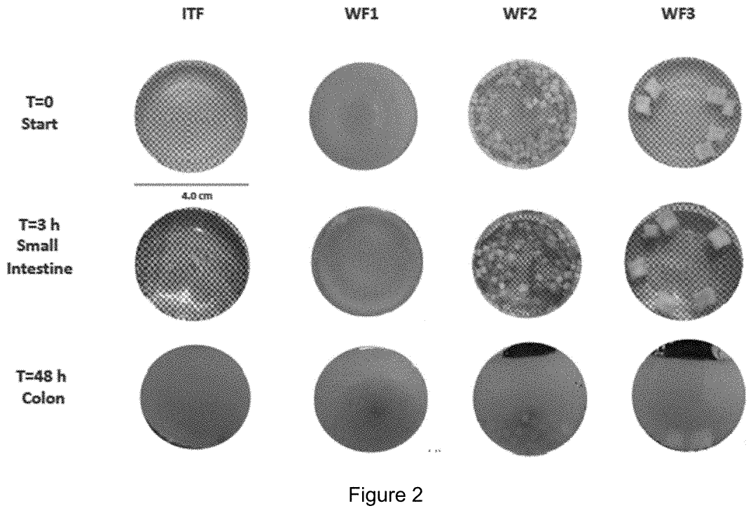

A crude dried chicory product was obtained by a process generally described in FIG. 2 of WO2014/172486 comprising steps 201, 202, 203, 204 and 207. A chicory root was washed and the top of the chicory root was cut off. The obtained product was heated in water containing ascorbic acid and CaCl2 at 50° C. for 60 minutes and was subsequently sliced. The sliced product was dried at 70° C. to obtain a crude dried chicory product.

WF1

The crude dried chicory product was ground to particles mostly having particle sizes of around 0.5 mm.

WF2

The crude dried chicory product was diced/ground to a lesser degree than in WF1 and was subsequently sieved to obtain particles which mostly consist of generally cubic particles having sides of around 2-3 mm (particle sizes of 2-4 mm).

WF3

The crude dried chicory product was diced/ground to a lesser degree than in WF2 and subsequently screened to obtain particles which mostly consist of generally cubic particles having sides of around 5-6 mm (particle sizes of 5-8

ITF

Extracted purified inulin powder with particles mostly smaller than 100 micrometer was obtained from a commercial source.

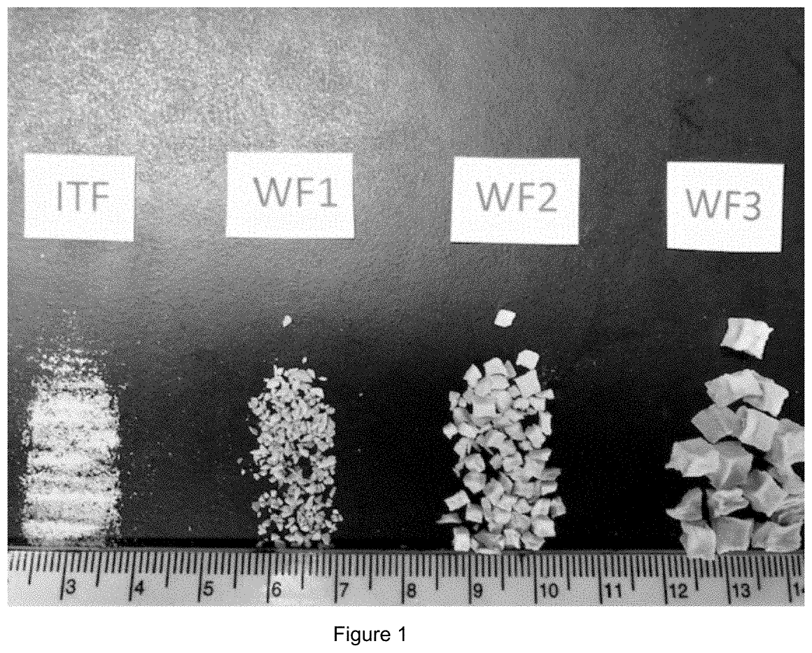

FIG. 1 shows photograph images of ITF and WF1-WF3, prior to the incubation as described below.

These inulin-containing products were tested in a gastro-intestinal model, for which an adapted SHIME system was used that consisted of stomach, small intestine and colon compartments as described in Marzorati et al., BMC Microbiol. 2014 May 22; 14:133 and van den Abbeele et al ISME J. 2013 May; 7(5):949-61. The latter was seeded by the fecal sample of a healthy middle-aged volunteer that was used for all incubations with the products (ITF, WF1, WF2 and WF3).

Visualization of the inulin-containing products at the start of the incubation in the simulated gastric compartment showed the characteristic size of the starting material. After three hours, the products show the characteristic size in the small intestinal transit. After 48 hours, the products show the characteristic size in the colonic compartment.

The results are shown in photograph images of FIG. 2.

ITF was readily soluble. WF1 showed some swelling. WF2 showed more swelling than WF1. WF3 showed more swelling than WF2.

After 48 h of incubation the concentration of the SCFAs acetate, propionate and butyrate were determined in triplicate incubations (Table 1).

TABLE 1
Production of SCFAs after 48 h of incubation. Entries are in mM or
ratios for butyrate/acetate (B/A) or butyrate/propionate (B/P).

| Product | Acetate | Propionate | Butyrate | Total | B/A | B/P |
|---|---|---|---|---|---|---|
| ITF | 34.9 | 24.9 | 4.1 | 63.9 | 0.11 | 0.16 |
| WF1 | 36.2 | 22.1 | 4.7 | 63.0 | 0.13 | 0.19 |
| WF2 | 35.8 | 21.1 | 5.1 | 62.0 | 0.14 | 0.24 |
| WF3 | 31.8 | 20.5 | 4.4 | 56.7 | 0.14 | 0.21 |

It can be understood that the product according to the invention (WF1, WF2 and WF3) generates short chain fatty acids (SCFAs) at a similar level and kinetics as the purified powder consisting essentially of inulin (ITF).

It can be further understood that the product according to the invention (WF1, WF2 and WF3) generates significantly higher levels of butyrate, resulting in a significantly higher ratio of butyrate over acetate (B/A) and butyrate over propionate (B/P), compared to the purified powder consisting essentially of inulin (ITF). Products with larger particle sizes (WF2 and WF3) show higher B/A ratio and B/P ratio than the product with smaller particle size (WF1). WF2 shows the highest B/A ratio and B/P ratio.

This indicates superior health benefits of the product of the invention since it has been well established that colonic butyrate has a panoply of health promoting functions.

Moreover, in the same period the increased pressure as a result of gas production was determined (Table 2).

TABLE 2
Gas production after incubation in the period 0-6 h,
6-24 h, 24-48 h and total period 0-48 h. Entries in kPa.

| Fiber | 0-6 h | 6-24 h | 24-48 h | 0-48 h |
|---|---|---|---|---|
| ITF | 11.5 | 71.7 | 11.2 | 94.4 |
| WF1 | 12.5 | 57.2 | 6.6 | 76.2 |
| WF2 | 10.7 | 58.3 | 6.5 | 75.6 |
| WF3 | 9.9 | 57.1 | 7.0 | 74.0 |

It can be further understood that the product according to the invention (WF1, WF2 and WF3) generates a lower level of gases compared to the purified powder consisting essentially of inulin (ITF). Comparison of WF1, WF2 and WF3 shows that the larger particle size leads to a lower level of gas generation.

Experimental Set 2

A randomized parallel investigator-blinded placebo-controlled trial was performed with 60 adults aged between 40-75 years, with fasting glucose levels between 5.6 and 6.9 mmol/L or 5.0-5.6 mmol/L and having a diabetes risk score≥9. These were randomly assigned to an intervention (n=30) or a control group (n=30). The subjects in the intervention group consumed 15 g per day of WF2 for 2 weeks and subsequently 30 g per day WF2 for 3 weeks while the placebo group consumed isocaloric amounts of maltodextrin.

Fasting blood glucose, fasting insulin and HOMA-ir decreased slightly in the WF2 intervention group after 5 weeks.

Moreover, faecal SCFA levels increased in the WF2 intervention group already after 2 weeks of 15 g WF2 consumption (increase 7.2 mM consisting of approximately 3.7 mM acetate, 1.3 mM propionate and 2.2 mM butyrate) and these were even further increased after the additional 3 weeks 30 g/per day WF consumption to 14.0 mM. No or only decreased changes in faecal SCFA levels were noted in the placebo group. The intake of WF2 improved stool consistency with an increase of 1.07 on the Bristol Stool Scale (p=0.0008) and increased stool frequency from 1.3 to 1.9 time per day (p=0.0002) as compared to the placebo.

This example demonstrates the effect of WF2 particles in increasing fecal SCFA levels and improving stool regularity and consistency. Of note is the observation that 15 g WF2 per day intake increases fecal SCFA levels and notably butyrate levels.

What is claimed is:

1. A vegetable product obtained from vegetable matter selected from at least one of root, tubers and leaves of a plant having inulin as a reserve carbohydrate, wherein
the product comprises particles having particle sizes in the range of 0.5 to 10 mm and comprising inulin and cell walls comprising pectin, hemi-cellulose and cellulose, wherein the inulin is contained within the cell walls, and
the product is obtained by a process comprising the steps of:
a) wounding the vegetable matter,
b) treating the vegetable matter with an aqueous solution comprising an antioxidant and/or a textural support agent and heating the vegetable matter to a temperature of 40 to 90° C. and
c) mechanically reducing the size of the vegetable matter and drying the vegetable matter to obtain the product.

2. The product according to claim 1, wherein the plant is selected from the group consisting of Jerusalem artichoke, globe artichoke, endive, Belgian endive, dandelion, chicory, dahlia, burdock, salsify and yacon.

3. The product according to claim 1, wherein the plant is chicory or Jerusalem artichoke.

4. The product according to claim 1, wherein at least 50 wt. % 60 wt. %, at least 70 wt.%, at least 80 wt. %, at least 90 wt. %, at least 95 wt. %, at least 98 wt. %, at least 99 wt. % or 100 wt. % of the product are the particles having particle sizes in the range of 0.5 to 10 mm.

5. The product according to claim 1, wherein the particles comprise particles having sizes in the range of 1.0 to 10 mm, preferably wherein at least 50 wt. % 60 wt. %, at least 70 wt. %, at least 80 wt. %, at least 90 wt. %, at least 95 wt. %, at least 98 wt. %, at least 99 wt. % or 100 wt. % of the product are the particles having particle sizes in the range of 1.0 to 10 mm.

6. The product according to claim 1, wherein the particles comprise particles having sizes in the range of 1.5 to 4.0 mm, preferably wherein at least 50 wt. % 60 wt. %, at least 70 wt. %, at least 80 wt. %, at least 90 wt. %, at least 95 wt. %, at least 98 wt. %, at least 99 wt. % or 100 wt. % of the product are the particles having particle sizes in the range of 1.5 to 4.0 mm.

7. The product according to claim 1, wherein the particles comprise particles having sizes in the range of 4.0 to 8.0 mm, preferably wherein at least 50 wt. % 60 wt. %, at least 70 wt. %, at least 80 wt. %, at least 90 wt. %, at least 95 wt. %, at least 98 wt. %, at least 99 wt. % or 100 wt. % of the product are the particles having particle sizes in the range of 4.0 to 8.0 mm.

8. The product according to claim 1, wherein at least 50 wt. %, at least 60 wt. %, at least 70 wt. %, at least 80 wt. %, at least 90 wt. %, at least 95 wt. %, at least 98 wt. %, at least 99 wt. % or 100 wt. % of the particles have a surface area to volume (SA/V) ratio of at most 6 mm⁻¹, 5 mm⁻¹, 4 mm⁻¹, 3 mm⁻¹, 2 mm⁻¹ or 1 mm⁻¹.

9. The product according to claim 1, wherein the amount of inulin with respect to the product is at least 50 wt. %, for example 60 to 80 wt. %.

10. The product according to claim 1, wherein the total amount of inulin, pectin and (hemi-)cellulose with respect to the product is at least 80 wt. %.

11. The product according to claim 1, wherein
the amount of inulin with respect to the product is 60 to 75 wt. %, e.g. 68 to 75 wt. %,
the amount of pectin with respect to the product is 5 to 12 wt. %, e.g. 7 to 12 wt. % and
the total amount of hemi-cellulose and cellulose with respect to the product is 4 to 8 wt. %,
preferably wherein the product further comprises protein, preferably in an amount of 4 to 7 wt. % with respect to the product.

12. A process for making a vegetable product obtained from vegetable matter selected from at least one of root, tubers and leaves of a plant having inulin as a reserve carbohydrate, wherein
the product comprises particles having particle sizes in the range of 0.5 to 10 mm and comprising inulin and cell walls comprising pectin, hemi-cellulose and cellulose, wherein the inulin is contained within the cell walls and
wherein the process comprises the steps of:
a) wounding the vegetable matter,
b) treating the vegetable matter with an aqueous solution comprising an antioxidant and/or a textural support agent and heating the vegetable matter to a temperature of 40 to 90° C.,
c) mechanically reducing the size of the vegetable matter and drying the vegetable matter to obtain the product.

13. The product according to claim 1 for use as a medicament.

14. The product according to claim 13 for use in one or more of the following:
Treatment of constipation and maintenance of normal defecation,
Treatment of metabolic and immune disorder selected from the group consisting of:
metabolic syndrome,
insulin-deficiency or insulin-resistance related disorders,
pre-diabetes,
Diabetes Mellitus (such as, for example, Type 2 Diabetes),
glucose intolerance,
abnormal lipid metabolism,
atherosclerosis,
hypertension,
cardiac pathology,
stroke,
non-alcoholic fatty liver disease,
hyperglycemia,
hepatic steatosis,
dyslipidemia,
dysfunction of the immune system associated with overweight and obesity,
cardiovascular diseases,
high cholesterol,
elevated triglycerides,
Increase of vaccination efficiency,
Increase of satiety,
Treatment and/or prevention of one or more of the following:
Antibiotic associated diarrhea
Acute gastroenteritis
Inflammatory Bowel Disease (IBD) including Ulcerative colitis and Chrohn's disease
Prevention of colorectal cancer
Treatment of distal colon cancer.

15. Use of the product according to claim 1 as a dietary supplement.
